(19) United States
(12) Patent Application Publication
(10) Pub. No.: US 2004/0142019 A1
(43) Pub. Date: Jul. 22, 2004

(54) Microbial-derived cellulose amorphous hydrogel wound dressing

(75) Inventors: Gonzalo Serafica, Langhorne, PA (US); Richard Mormino, San Antonio, TX (US); Russell A. Hoon, Doylestown, PA (US)

Correspondence Address: FOLEY AND LARDNER, SUITE 500, 3000 K STREET NW, WASHINGTON, DC, 20007, US

(73) Assignee: Xylos Corporation

(21) Appl. No.: 10/345,394

(22) Filed: Jan. 16, 2003

(51) Int. Cl. A61L 15/00; A61L 15/16

(52) U.S. Cl. 424/445

(57) ABSTRACT

A microbial-derived cellulose wound dressing is provided which is in the form of a hydrogel which can be used to treat chronic wounds and burns.

### FIELD OF THE INVENTION

The invention relates to a wound dressing comprising microbial-derived cellulose in an amorphous hydrogel form.

### BACKGROUND OF THE INVENTION

There are numerous wound dressings that demonstrate effectiveness to aid in the healing of wounds. The components of these include various polymeric systems, cellulosic materials derived from plants and bacteria, and collagen. Each has its mode of action to assist the wound healing process. Many rely on either the donation of fluid to hydrate a wound surface and aid in removal of necrotic tissue through autolytic debridement or the absorption of excess fluid termed exudate.

Microbial-derived cellulose dressings are composed of bacterial cellulose and water. The processing of which, results in a dressing that possesses unique characteristics. Not only can it donate moisture which is associated with the dressing but its multi-layered three-dimensional structure, that distinguishes it from plant-derived cellulose, creates a material with a water-holding capacity up to 700 times its own dry weight, as described in U.S. Pat. No. 4,942,128. Microbial cellulose also demonstrates excellent wet tensile and compression strength. Lastly, by adjusting the cellulose to liquid ratio in processed microbial cellulose, the amount and rate of both fluid donation and absorption can be manipulated.

Because of its superior characteristics, use of microbial cellulose in the medical industry has been previously investigated. For example, U.S. Pat. Nos. 4,588,400, 4,655,758 and 4,788,146 to Ring et al. disclose the possible use of microbial-derived cellulose in liquid-loaded medical pads. The patents to Ring et al focus on using statically produced microbial cellulose pads loaded with various liquids and medicaments. These pads were detailed as well as the production and cleaning method to produce the starting cellulose material. Also described in these patents are examples detailing methods of fabrication of various pads, wherein the method involves a series of pressing and soaking steps to adjust the physical properties, mainly with respect to the liquid to cellulose ratio, to yield a desired product. As an example, these patents illustrate a highly hydrated pad (80 to 1 fluid to cellulose ratio) that is able to provide a cooling capability ideal for bum applications. In particular, the '146 patent describes the use of such liquid loaded pads as wet dressings for use as an ulcer dressing capable of providing moisture to the wound over an extended period of time. The same '146 patent mentions that the wet dressings described in the examples have the additional ability to absorb large quantities of fluid from the wound site when the dressing is applied in a less than saturated condition. However, the wound dressings of Ring et al. fail to mention a singular dressing having both the ability to be a source of moisture for wounds as well as the ability to absorb fluid. The Ring et al. patents also fail to describe the effective liquid to cellulose ratio to fabricate a dressing having the dual fluid handing capability. Furthermore, the Ring et al. patents do not describe microbial-derived cellulose wound dressings in an amorphous gel form.

Amorphous hydrogel dressings, for example IntraSite Gel (Smith & Nephew), differ from other dressings in their ability to add moisture to a dry wound and as such have been shown to be useful for debriding necrotic dry tissue found in chronic and bum wounds. Since these hydrogels have not been cross-linked and therefore do not take a fixed shape, they have been termed amorphous (Ovington, L. G., Amorphous Gels Can Help Dry Escharic Wounds, Wound Care Institute Newsletter, July/August 1997, Volume 2, No. 3).

Rhodes, in U.S. Pat. No. 5,662,924, describes a wound dressing that contains a water-insoluble water swellable cross-linked cellulose derivative, water and a polyol. This dressing, in the form of an amorphous gel, is believed to enhance moisture penetration of necrotic tissue and thereby facilitate wound healing by speeding up debriding action.

The present inventors have developed a flowable cellulose-based gel wound dressing that possesses this novel fluid handling capability of absorption and donation. Surprisingly, production of a microbial-derived cellulose wound dressing in an amorphous gel form enhances the moisture donating aspect of the wound dressing relative to the unprocessed microbial cellulose starting film material. This fluid handling capability is an end result of the processed microbial cellulose that contains the proper cellulose content for the intended purpose. The resulting wound dressing can donate fluid if the wound surface is dry and found to be particularly useful for dry wounds covered with dry necrotic tissue or eschar. Here it acts to autolytically debride the wound: the necessary first step in healing of a chronic wound.

Surprisingly, at the optimal cellulose content, the same dressing is also capable of absorbing fluid away from the exuding wound bed. Typically, chronic wounds such as venous ulcers tend to exude large amounts of fluids during the healing process. At this stage the dressing of the present invention is able to absorb the fluid exudate while maintaining a moist surface for epithelial cells to migrate. The epithelial migration is essential for eventually closing the wound.

Furthermore, the flowable nature of this material allows this dressing to fill areas that a pad cannot effectively treat. The amorphous gel dressing can be delivered to the entire wound bed surface. The intimate contact of the gel dressing with the entire wound surface further enhances the moisture donation and absorption quality of microbial-derived cellulose and thereby improves wound healing. When it is necessary to change the dressing the amorphous gel dressing can be easily removed without upsetting the newly forming tissue. Also, since it can be removed en bloc, the wound cleansing process, required for other gel dressing products, is greatly simplified.

### SUMMARY OF THE INVENTION

It is an object of the present invention to provide microbial-derived cellulose wound dressings in an amorphous gel form, comprising 1% to 10% cellulose by weight. In a preferred embodiment, the microbial-derived cellulose is biocompatible and nonpyrogenic.

It is another object of the present invention to provide an effective wound dressing comprising microbial cellulose in an amorphous gel form that is capable of enhanced donation of moisture for improved wound healing.

Further, it is an object of the present invention to provide an effective wound dressing comprising microbial cellulose that can flow to fill an area and then be easily removed when changing is necessary.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE PREFERRED EMBODIMENT

Unless otherwise specified, “a” or “an” means “one or more”.

The preferred biosynthesized cellulose for the amorphous gel is produced by cellulose-producing organisms, such as Acetobacter xylinum, and is subjected to a series of chemical wash steps to render it non-pyrogenic. Once grown the typical processing uses hydroxide solutions at concentrations of 0.5-20% by weight. Preferably, sodium hydroxide is used at a concentration of not less than 1% by weight and most preferably about 2% to about 4% by weight in order to dissolve the cells. In addition, the present invention provides hydrogen peroxide washing capable of whitening and sanitizing the non-pyrogenic films.

Cellulose pellicles are typically composed of greater than 98% water and from 0.2 to 2% cellulose by weight. Subsequent to chemical processing, the pellicles are wet milled to produce the amorphous gel form with a cellulose content roughly equivalent to that of the starting material but which can be adjusted to any desired concentration through the addition or removal of fluids. The amorphous gel wound dressing obtained from the milling and grinding of the intact microbial cellulose pellicles has a primary structure of ultra fine fibers that are known to be about 200 times finer than cotton fibers. The secondary structure, which is a non-woven pattern of interpenetrating cellulose fibers, is also not completely disrupted.

Typical cellulose content of the present invention ranges from about 1.0% to about 99% cellulose by weight, preferably about 2.5% to 65% by weight, more preferably about 3.0% to 50% by weight and most preferably 3.5% to about 12% by weight. In an especially preferred embodiment, the cellulose content is about 4% or about 7% by weight.

The amorphous gel dressings of the invention can be used for donation of liquid to wounds as well as absorbing liquid from wounds. Typically, the microbial-derived cellulose dressing can donate between about 40 to 85% of its liquid weight and can absorb between about 10 to 50%, more preferably the dressing can donate about 50 to 65% of its liquid weight and absorb about 15 to 35% of its weight in liquid.

The flowable nature of the wound dressing can be manipulated by the addition of an ingredient for flow modification. Such ingredients include but are not limited to polyols. The polyols include propylene glycol, glycerol, polyethylene glycol and sorbitol and the like.

The rheological properties of the gel are easily adjusted by addition of liquids or solids such as polyols, i.e., polyethylene glycol, sorbitol, mannitol, glycerol, and propylene glycol or other flow modification agents such as lecithin and aloe vera. The concentration of these additives in the microbial cellulose gel may vary from 1% to 50% by weight depending on the properties of the specific additive and on the desired flow characteristics of the resulting gel.

Liquid materials which can be loaded into the gel include but are not limited to water, isotonic saline, synthetic polymers such as polyethylene oxide, polyvinylpyrrolidone, aqueous solutions of molecules including proteins, such as platelet derived growth factor (PDGF), epidermal growth factor (EGF), fibroblast growth factor (FGF), insulin-like growth factor (IGF), Transforming growth factor-beta (TGF-β), bone morphogenetic protein (BMP), vascular endothelial growth factor (VEGF), nerve growth factor (NGF), tumor angiogenesis factor (TAF), corticotropin releasing factor (CRF), interleukin-8 (IL-8), granulocyte-macrophage colony stimulating factor (GM-CSF), and other growth factors, and enzymes such as collagenase, papain and fibrinolysin desoxynuclease. Additionally the dressing may contain one or more active agents like antibiotics, such as bacitracin, polymyxin B, gentamicin, chloramphenicol, mupirocin, neomycin, silver sulfadiazine, gramicidin, and the like: topical anesthetics, such as lidocaine hydrochloride, benzocaine, dibucaine, tetracaine hydrochloride and the like: antifungal agents, such as clotrimazole, econazole, ketoconazole, miconazole, nystain, terbinafine, tolnaftate, undecylenic acid and the like; antiseptics and preservatives, such as polyhexamethylene biguanide, chlorhexidine digluconate, benzalkonium chloride, silver-based antimicrobials, copper-based antimicrobials and the like; antiviral agents, such as gentamycin sulfadiazine, dapsone, ampicillin, amphotericin B, silver halides, silver protein, colloidal silver, erythromycin and the like.

Compared to the intact microbial cellulose pellicles, the amorphous gel form can be formulated to enhance the donation and/or absorption characteristics of the gel. The content of microbial-derived cellulose present in the amorphous gel dressing can be manipulated depending upon the method of preparation and the eventual end use of the wound dressing.

The present invention also relates to a method of treatment of wounds using the inventive wound dressing. In a preferred embodiment, chronic wounds or burns are treated with the inventive wound dressing. The method comprises applying the wound dressing to the wound site, filling the wound with the hydrogel dressing, and covering the wound with a secondary film layer. The frequency of changing the dressing is readily determined by one skilled in the art. In one embodiment, the dressing is changed twice daily to weekly.

The present invention will be illustrated through the following examples.

### EXAMPLE 1

### 1. Production of Microbial Cellulose

In preparing the microbial cellulose amorphous gels of the invention, a microbial cellulose film is prepared. The film is prepared by using microorganisms such as Acetobacter xylinum which are cultured in a bioreactor containing a liquid nutrient medium at 30 degrees ° C. at an initial pH of 3-6. The medium is based on sucrose or other carbohydrates. Preferably, efficient film production is achieved using sucrose as a carbon source, ammonium salts as a nitrogen source, and corn steep liquor as nutrient source coupled with a proprietary trace elements supplement, which varies from the original Schramm & Hestrin medium (1954) used by those skilled in the art. This proprietary trace elements supplement is quantified in the following table:

Trace Element Solution

Composition Per Liter

| | | |
|---|---|---|
| EDTA | Tetrasodium Salt | 570 mg |
| FeSO₄ | 7H₂O | 200 mg |
| ZnSO₄ | 7H₂O | 10 mg |
| MnSO₄ | H₂O | 26 mg |
| H₃BO₃ | | 30 mg |
| CoCl₃ | 6H₂O | 20 mg |
| NiCl₂ | 6H₂O | 3.2 mg |
| (NH₄)₆Mo₇O₁₄ | 4H_{2kp[}O | 2.4 mg |

Two ml of this solution is added per liter of media.

Suitable bioreactors are selected which minimize evaporation and provide adequate oxygen-limiting conditions. Oxygen-limiting conditions may be varied depending upon the desired water content and thickness of the cellulose film. Generally, under oxygen-limited conditions, oxygen is present in an amount of 5%-21% of the total gas present at the air liquid interface. The bioreactor is composed of plastic box fitted with an airtight cover or a limited gas-permeable cover. Dimensions of the bioreactor can vary in configuration (cube or cylinder) depending on the shape and size of the cellulose film being produced. For example, a six inch diameter cylinder will produce a six inch diameter dressing, which can be used as is or cut to conform to the wound to be treated, prior to application. By limiting the amount of oxygen in the fermentation medium, it is hypothesized that the Acetobacter utilizes the carbon available in the medium to produce more cellulose instead of using it for reproduction, thereby increasing the total yield of cellulose.

The fermentation process under static conditions was allowed to progress over for a period of about 7-30 days, during which the bacteria in the culture medium produced an intact cellulose pellicle containing the microorganisms. Depending on the desired thickness, which corresponds to a certain cellulose content per unit area, the fermentation is stopped and the pellicle is removed from the bioreactor. The excess medium contained in the pellicle is then removed by standard separation techniques such as compression or centrifugation prior to chemical cleaning and subsequent processing of the pellicle to yield a wound dressing with a cellulose to liquid ratio of about 1:10 to about 1:65. The raw cellulose pellicle has an increased sugar:cellulose yield of about 35%, compared to literature values of 10%. This increased yield coupled with an inexpensive nitrogen source resulted in a 40-fold reduction in production-cost of the raw cellulose film as compared to cellulose films produced according to the original Schramm & Hestrin medium [1954, J. Gen. Micro, 11:123-129].

### 2. Processing and Depyrogenation Procedures

After the cellulose film has been produced, the cells have to be removed from the cellulose pellicle for purification. Fontana et al. (1990, Appl. Biochem. Biotech, 24: 253-264) have described the cells as being apyrogenic, however, the unpurified cellulose pellicle has tested positive for pyrogens using the Limulus Amebocyte Lysate (LAL) test kit. This result necessitated the removal of the cells by chemical processing discussed here in order to pass the standard pyrogenicity test and qualify the microbial cellulose wound dressing as nonpyrogenic.

The cellulose pellicle is subjected to a series of chemical wash steps to convert the raw cellulose film into a medical grade and non-pyrogenic wound dressing material. Typical processing uses hydroxide solutions at concentrations of 1-20% by weight. Preferably, sodium hydroxide is used at a concentration of not less than 3% and most preferably about 3% to about 5% in order to dissolve the cells. In addition, the present invention provides hydrogen peroxide washing capable of bleaching and sterilizing the pyrogen-free films. Concentrations of about 0.05% to about 10% peroxide by weight are useful to effect whitening of the films. Preferably the amount of peroxide used in about 0.1% to about 0.5%. Other bleaching agents such as hypochlorite, hypobromite, and perborate may also be used.

Purification processes using various exposure times, concentrations and temperatures were conducted on the raw fermentation product. Processing times of 1-4 hours have been studied in conjunction with temperature variations of 30-100 degrees centigrade to optimize the process. The resulting films from each of the different operating conditions were tested for their respective pyrogen levels and physical characteristics. The process condition that yields a nonpyrogenic product in the least amount of time and lowest chemical concentration was then selected for economic reasons. The time involved in this process can be as much as 4 hours at about 90° C., preferably the time involved is about 1-2 hours at about 60° C. to about 80° C.

The amount of cellular debris left in the cellulose pad after processing may be measured by Limulus Amebocyte Lysate (LAL) test as outlined by the U.S. Food and Drug Administration (FDA) in 21 CFR10.90. The instant cleaning process outlined above provided a nonpyrogenic cellulose pad (<0.05 EU/ml). The allowable pyrogen content in Class I medical devices is 0.5 EU/ml (FDA LAL test Guideline). The steps of the LAL test are defined by the test kit manufacturer and can simply be followed to yield the pyrogen level in the cellulose film.

### EXAMPLE 2

### Production of a Microbial Cellulose Amorphous Gel

This example presents a method for making an amorphous gel material from microbial cellulose sheets. The cellulose sheets were processed using the method described in Example 1 to remove pyrogens and other contaminants, and compressed to obtain a cellulose content of approximately four percent.

A 500 g quantity of the processed and depyrogenated microbial cellulose was placed in a 1 gal blender. To this 2500 ml of deionized water was added, and the mixture was processed using a 3 hp motor at high speed for 5 min to ensure consistency. The resulting mixture was decanted into a draining bin, and excess water was allowed to drain. After draining for 15 min, the mixture was pressed until the weight of the gel again reached 500 g.

Two 20 g samples of the gel were removed and dried to determine the cellulose content of the gel. The average dry weight was 0.85 g, indicating a cellulose content of 4.25% by weight

### EXAMPLE 3

### Modification of Flow Properties

This example demonstrates how the viscosity and flow properties of a microbial cellulose amorphous gel can be modified through the addition of an ingredient for flow modification.

Amorphous gel was produced by the method described in example 1, and the final cellulose content was determined to be 3.95% by drying of 20 g aliquots. Using this gel, nine 50 g samples were prepared containing 0 to 40 percent propylene glycol by weight. The gels were mixed thoroughly to distribute the propylene glycol and then packed into identical 5 cc disposable syringes with 1.5 mm tip openings.

The maximum force required to discharge the material from the syringes was measured with a compact force gauge and was plotted versus the propylene glycol content. This image is shown in FIG. 1. The discharge force initially dropped rapidly with the addition of the flow modifying agent, but the cumulative effect diminished as the concentration was increased. At around 25% propylene glycol the force leveled off at 4.5 N, with higher concentrations showing no discernible effect.

### EXAMPLE 4

### Addition of Active Agents

This example shows how the properties of a microbial cellulose amorphous gel can be changed through the addition of active agents. The amorphous gel used for this example was produced using the method described in example 1.

The 500 g gel was divided in half. The first half was modified with the addition of polyhexamethylene biguanide (PHMB) in sufficient quantity to give a 0.25% concentration. The second half of the gel was kept unchanged. Both gels were sterilized by gamma irradiation at 30-35 kGy. The samples then underwent antimicrobial testing. 10 g samples of each gel were inoculated with 10⁵ cultures of either Staphylococcus aureus or Esherichia coli and incubated at 30° C. Organism populations were measured at time zero and again after 24 hr, and the totals of the PHMB-treated gel were compared with the untreated control.

Results:

TABLE 1
Bacterial Inhibition by PHMB-Containing Amorphous Gel

| | Population Count (cfu/ml) | | | |
|---|---|---|---|---|
| | Time 0 | | Time 24 hr | |
| Sample | S. aureus | E. coli | S. aureus | E. coli |
| PHMB-treated | 1.5 × 10⁵ | 2.0 × 10⁵ | <10 | <10 |
| Untreated | 1.5 × 10⁵ | 2.0 × 10⁵ | 3.7 × 10² | 6.2 × 10⁴ |

The amorphous gel treated with 0.25% PHMB reduced the bacterial population of both species by 99.99%, whereas the untreated amorphous gel resulted in significantly less reduction.

### EXAMPLE 5

### Preparation of an Amorphous Gel Wound Dressing

This example demonstrates the method of producing a wound dressing comprised of microbial cellulose amorphous gel. This dressing will have the ability to both donate moisture to or absorb moisture from a wound site, depending on the state of the wound.

Amorphous gel was produced following the method described in example 1. Using the 500 g gel as a base material and assuming the initial cellulose content to be 4%, eight samples were created ranging from 1 to 10 percent cellulose according to the following table:

TABLE 2
Composition of Amorphous Gel Samples

| % | | | | | % |
|---|---|---|---|---|---|
| Cellulose | Mass of | Water (g) | | | Cellulose |
| (assumed) | Gel (g) | Addition | Subtraction | Total Weight | (actual*) |
| 1 | 12.5 | 37.5 | — | 50 | 1.17 |
| 2 | 25.0 | 25.0 | — | 50 | 2.41 |
| 3 | 37.5 | 12.5 | — | 50 | 3.42 |
| 4 | 50.0 | — | — | 50 | 4.71 |
| 5 | 62.5 | — | 12.5 | 50 | 5.48 |
| 6 | 75.0 | — | 25.0 | 50 | 6.39 |
| 8 | 100 | — | 50.0 | 50 | 8.87 |
| 10 | 125 | — | 75.0 | 50 | 11.1 |

*A sample of each was dried to determine the exact cellulose content.

These samples were then tested for absorption from a saturated sponge and donation to a dry surface. For the absorption test, a 5 g sample of the gel was spread evenly over a 2 in circular area on a sheet of filter paper. The paper was placed on top of a sponge sitting in a 0.9% saline bath at room temperature. The liquid level was maintained at the level of the sponge. Samples were removed after 24 hr and reweighed to determine the quantity of saline absorbed by the gel, and the absorption was reported as a percentage of the initial weight of the sample. FIG. 2 shows the absorption profile for this set of amorphous gels. As can be seen, gels containing less than 3% cellulose lost weight during the test, indicating that moisture was donated to the wet sponge. The inflection point on the curve occurred at approximately 5.5% cellulose by weight, and increased rapidly from there as the cellulose content increased.

Donation testing was performed by spreading a 5 g sample of gel evenly over a 2 in diameter circular area on a 3 in×3 in piece of pre-weighed smooth leather. Samples were removed after 2 hr and the leather was reweighed to determine the quantity of moisture donated to the dry surface. Donation results were reported as a percentage of the initial weight of the sample, and are shown graphically in FIG. 3. Donation decreased nearly linearly up to 6% cellulose by weight, and then decreased more slowly up to the 11% by weight.

Using FIGS. 2 and 3, a wound dressing can be devised to accommodate both absorption and donation. In order to have measurable absorption, the gel would need to possess a minimum of 4% cellulose, and the gel would need less than 6% cellulose to donate significantly. Therefore, a wound dressing gel should contain between 4 and 6 percent cellulose to optimize the natural fluid handling ability of the microbial cellulose matrix.

Each of the references cited above is incorporated herein in its entirety to the same extent as if each reference was individually incorporated by reference.

Though the invention has been described with reference to particular embodiments, it is recognized that variations and equivalents of these embodiments may be used without departing from the scope or spirit of the invention.

What is claimed is:

1. A microbial-derived cellulose amorphous gel wound dressing comprising a cellulose content by weight selected from the group consisting of about 1.0% to about 99%, about 2.5% to 65%, about 3.0% to 50%, 3.5% to about 12%, 4% and 7%.

2. The wound dressing of claim 1, comprising about 4% or 7% cellulose.

3. The wound dressing of claim 1, further comprising an ingredient for flow modification.

4. The wound dressing of claim 1, further comprising a preservative.

5. The wound dressing of claim 1, further comprising one or more active agents.

6. The wound dressing of claim 3, wherein the ingredient for flow modification is a polyol.

7. The wound dressing of claim 6, wherein said polyol is present in the dressing from about 5 to about 50 wt % and is selected from the group consisting of propylene glycol, glycerol, polyethylene glycol and sorbitol.

8. An amorphous gel dressing of claim 4, wherein the preservative is one or more of the following group: chlorhexidine digluconate, polyhexamethylene biguanide hydrochloride or silver compounds.

9. An amorphous gel dressing of claim 5, wherein the one or more active agents are selected from the group consisting of antimicrobials, antibiotics, antivirals, enzymes, proteins and growth factors.

10. An amorphous gel dressing of claim 9, wherein the antibiotic, antimicrobial or antiviral active agent is selected from the group consisting of bacitracin, polymixin B, gentamicin, chloramphenicol, mupirocin neomycin, silver sulfadizine, gramicidin, ofloxicin, tetracycline, streptomycin, fluoroquinolones, ganciclovir, acyclovir,clindamycin, clortimazole, econazole, ketoconazole, miconazole, nystain, terbinafine, tolnaftate, undecylenic acid, gentamycin sulfadiazine, dapsone, ampicillin, amphotericin B, silver halides, silver protein, colloidal silver and erythromycin.

11. An amorphous gel dressing of claim 9, wherein the enzymes, proteins and growth factors are selected from the group consisting of collagenase, papain, fibrinolysin, desoxyribonuclease, platelet derived growth factor(PDGF), epidermal growth factor (EGF), acidic and basic fibroblast growth factors (FGF-1 and FGF-2), insulin-like growth factors 1+2(IGF-1 and IGF-2), vascular endothelial growth factor (VEGF), nerve growth factor (NGF), tumor angiogenesis factor (TAF), corticotropin releasing factor (CRF), interleukin-8 (IL-8), granulocyte-macrophage colony stimulating factor (GM-CSF), transforming growth factors alpha and beta (TGF-alpha and TGF-beta), bone morphogenetic protein (BMP), interferons, interleukins and albumin.

12. The amorphous gel dressing of claim 1, where the microbial-derived cellulose dressing donates 40 to 85% of its liquid weight and absorbs 10 to 50% of its weight.

13. The amorphous gel dressing of claim 1, wherein the microbial-derived cellulose dressing donates 50 to 65% of its liquid weight and absorbs 15 to 35% of its weight.

14. A method for preparing a microbial-derived cellulose amorphous gel wound dressing comprising:
production of a microbial cellulose pellicle;
isolation of a pellicle with a cellulose content by weight in the range of about 0.5 to about 1%; and
wet milling the pellicle to produce an amorphous gel with a cellulose content by weight of 0.5% to 5%.

15. The method as claimed in claim 14, wherein the microbial cellulose pellicle is obtained from Acetobacter xylinum.

16. A method for treating chronic wounds or burns comprising: applying a nonpyrogenic, biocompatible microbial-derived cellulose amorphous gel wound dressing to a wound site.

17. A method as claimed in claim 16, further comprising
filling the wound with the gel dressing,
covering with a secondary film dressing, and
changing the cellulose gel dressing from twice daily to weekly,
wherein said microbial-derived cellulose amorphous gel dressing comprises a cellulose content selected from the group consisting of about 1.0% to about 99%, about 2.5% to 65%, about 3.0% to 50%, 3.5% to about 12%, 4% and 7%.

18. The method of claim 16, wherein the microbial-derived cellulose amorphous gel dressing further comprises an ingredient for flow modification.

19. The method of claim 16, wherein the microbial-derived cellulose amorphous gel dressing further comprises a preservative.

20. The method of claim 16, wherein the microbial-derived cellulose amorphous gel dressing further comprises one or more active agents.

21. A method of claim 18, wherein the ingredient for flow modification is present in the dressing about 5 to about 50 wt % and is a polyol selected from the group consisting of propylene glycol, glycerol, polyethylene glycol and sorbitol.

22. A method of claim 19, wherein the preservative is at least one selected from the group consisting of chlorhexidine digluconate, glycerol monolaurate or polyhexamethylene biguanide hydrochloride.

23. A method of claim 20, wherein the one or more active agents are selected from the following groups: antimicrobials, antibiotics, antivirals, enzymes, proteins and growth factors.

24. A method of claim 23, wherein the antibiotics, antimicrobial or antiviral are selected from the group consisting of bacitracin, polymixin B, gentamicin, chloramphenicol, mupirocin, neomycin, silver sulfadizine, gramicidin, ofloxicin, tetracycline, streptomycin, fluoroquinolones, ganciclovir, acyclovir,clindamycin, clortimazole, econazole, ketoconazole, miconazole, nystain, terbinafine, tolnaftate, undecylenic acid, gentamycin sulfadiazine, dapsone, ampicillin, amphotericin B, silver halides, silver protein, colloidal silver and erythromycin.

25. A method of claim 23, wherein the enzymes, proteins and growth factors are selected from the group consisting of collagenase, papain, fibrinolysin, desoxyribonuclease, platelet derived growth factor (PDGF), epidermal growth factor(EGF), acidic and basic fibroblast growth factors (FGF-1 and FGF-2), insulin-like growth factors 1+2 (IGF-1 and IGF-2), vascular endothelial growth factor (VEGF), nerve growth factor (NGF), tumor angiogenesis factor (TAF), corticotropin releasing factor (CRF), interleukin-8 (IL-8), granulocyte-macrophage colony stimulating factor (GM-CSF), transforming growth factors alpha and beta (TGF-alpha and TGF-beta), bone morphogenetic protein (BMP), interferons, interleukins, and albumin.

26. A method of claim 16, wherein the microbial-derived cellulose dressing donates 40 to 85% of its liquid weight and absorbs 10 to 50% of its weight.

27. The method of claim 16, wherein the microbial-derived cellulose dressing donates 50 to 65% of its liquid weight, while absorbing 15 to 35% of its liquid weight.
